(19) United States
(12) Patent Application Publication
(10) Pub. No.: US 2007/0027079 A1
(43) Pub. Date: Feb. 1, 2007

(54) GHRP 2 strips

(76) Inventors: Lorn Leitman, Key Biscayne, FL (US); Gustavo Barni, Pompano Beach, FL (US)

Correspondence Address: Lorn Leitman, 325 SW 15 Avenue, Pompano Beach, FL, 33069, US

(21) Appl. No.: 11/189,962

(22) Filed: Jul. 27, 2005

(51) Int. Cl. A61K 38/27

(52) U.S. Cl. 514/12

(57) ABSTRACT

The products that exist on the market today that produce the same results are with injection. This new product, HGH Strips, is taken orally through strips that are placed on the back of the toungue. HGH Strips is scientifically developed with GHRP-2 (Growth Hormone Releasing Peptide-2), L-Lysine, L-Argenin and L-Valine which amino acids are specifics for increasing the natural production of GH (Growth Hormone) by the anterior pituitary deep inside the brain; also called Somatotropin, it influences in growth of cells, bones, muscles and organs throughout the body. HGH Strips helps to increasing the levels of IGF-1 (Insulin like Growth Factor-1 is a metabolite of human growth hormone) rise; a natural anabolic growth factor molecule. Studies have found that IGF-1 has increased lean body mass, reduced fat, built up bone and muscle tissue, it may even regenerate nerve tissues and boost the immune system.

### INTRODUCTION

GHRP-2 is a type of Growth Hormone Releasing Peptide. It is a fragment of Human Growth Hormone (HGH)

Growth Hormone Releasing Peptides (GHRP) have been demonstrated to significantly increase the IGF-1 levels in humans as they mature and become older.

The product, HGH STRIPS, is an all-natural secretagogue that is designed to stimulate the pituitary gland to release human grown hormone (HGH).

HGH STRIPS is an exciting scientific breakthrough that may help to slow and in some cases reverse the aging process. Thankfully, it is now possible and probable that we can now slow the aging process.

What can HGH STRIPS help promote?
Increase Energy/Vitality
Reduce Fat/Cellulite
Build Muscle
Rejuvenate Skin, Hair and Nails
Improve Memory
Elevate Mood
Enhance Sexual Desire
Regulate Sleep
Rejuvenate the Immune System
Improve Brain Function
Protect Cells

### 1. Production Diagram of HGH Strips

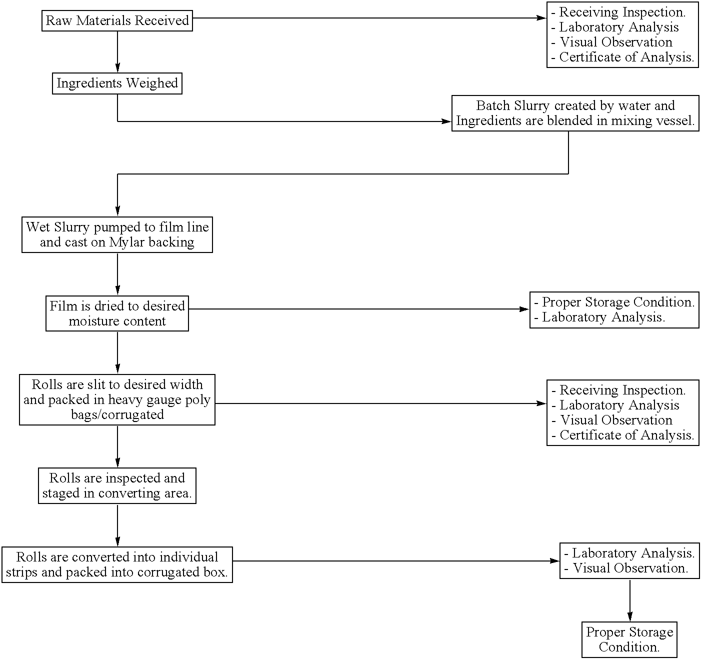 CERTIFICATE OF ANALYSIS
GHRP-2

| | |
|---|---|
| Name of Product: | GHRP-2 (C₄₅H₅₅N₉O₆) |
| | D-Ala-D-Nal-Ala-Trp-D-Phe-Lys-NH2 |

| | | | |
|---|---|---|---|
| Batch No: | 6P-20030403 | Analyzed On: | 4/3/2003 |
| Date of Manufacture: | 4/2/2003 | Quantity: | 5.015 g |

| TEST | | SPECIFICATION | RESULTS |
|---|---|---|---|
| 1. | Appearance | White to off-white, amorphous powder of low | Complies |
| | | density | |
| 2. | Solubility | Soluble in water and 1% acetic acid at a | Complies |
| | | concentration of ≧1 mg/ml to give a clear, | |
| | | colorless solution. | |
| 3. | Identity | Monoisotopic Mass: 817.4 ± 1 | 817.2 |
| 4. | Amino Acids | Ala: 1.7-2.3 | 1.88 |
| | | Lys: 0.85-1.15 | 1.02 |
| | | Phe: 1.7-2.3 | 1.85 |
| | | Trp: Present | Present |
| 5. | Peptide Purity | ≦95.0% | 96.08% |
| | (By HPLC) | | |
| 6. | Assay | ≦80.0% | 83.70% |
| | (By Peptide Content) | | |
| 7. | Acetic Acid | ≦15.0% | 12.70% |
| 8. | Water (K.F.) | ≦10.0% | 3.60% |
| 9. | Methylene Chloride | ≦0.1% | 0% |
| | (By GC) | | |
| 10. | Acetonitrile | ≦0.1% | 0% |
| | (By GC) | | |
| 11. | Fluoride | ≦0.01% | 0.00% |

CERTIFICATE OF ANALYSIS
FINAL PRODUCT

| Product Description | | | Lot No: | G1-531-05 |
|---|---|---|---|---|
| Product: | HIGH STRIPS | | Manufacture Date: | 6/24/2005 |
| Dosage form: | Strips | Serving Size: 3 | | |
| Size: | 1.25″ × .875″ | Serving per Container: 10 | Expiration Date: | 6/24/2008 |

PHYSIC-CHEMICAL TEST

| TEST | SPECIFICATIONS | RESULT | METHOD |
|---|---|---|---|
| Desintegration | NMT 1 min | 16 seconds | USP 25 <2040> |
| Weight Variation | 41.5 mg +/− 5% | 43.575 mg | USP 25 <2091> |
| Appearance | Strips | Complies | Visual |
| Color | Tan | Complies | Visual |
| Flavor | Peppermint | Complies | Taste |
| Odor | Characteristic | Characteristic | Organoleptic |

| | Lower | | Upper | | Label | | Assay* | | % Label |
|---|---|---|---|---|---|---|---|---|---|
| Active Ingredient | Limit | U | Limit | U | Claim | U | Result | U | Claim |
| Proprietary Blend: | 90 | % | 110 | % | 8.5 | mg | 8.5 | mg | 100% |
| (L-Lysine, L-Arginine, L-Valine and GHRP-2*) | | | | | | | | | |

*GHRP-2 (D-Ala-D-Nal-Ala-Trp-D-Phe-Lys-NH2)
*By Input

MICROBIOLOGICAL TEST

| TEST | SPECIFICATIONS | RESULTS | METHOD |
|---|---|---|---|
| Total Viable Count | NMT 3.000 cfu/g | <3.000 cfu/g | USP 25 <2021> |
| Total Molds & Yeasts | NMT 300 cfu/g | <300 cfu/g | USP 25 <2021> |
| Salmonella | Negative | Negative | USP 25 <2021> |
| E. Coli | Negative | Negative | USP 25 <2021> |
| S. Aureus | Negative | Negative | USP 25 <2021> |
| Pseudomonas | Negative | Negative | USP 25 <2021> |

### HGH (Human Growth Hormone)

HGH (Human Growth Hormone) is referred to in medical science as the main hormone. It is very plentiful when we are young, but near the age of twenty-one our bodies begin to produce less of it. By the time we are forty nearly everyone is deficient* (please see following pages for more explanation on HGH decrease with age) in HGH, and at eighty our production has normally diminished at least 90-95%.
*These statements have not been evaluated by the Food and Drug Administration. This product is not intended to diagnose, treat, cure, or prevent any disease.

Hormones are chemical messengers, traveling through the blood stream and involved in all aspects of the body's activity—metabolism, growth, mood and sexual function to name a few. They also help to maintain health, regulate the vital organs, and promote healing and repair.

Being the main hormone, HGH affects virtually all areas of the body—influencing the growth of cells, bones, muscles and organs. When we become deficient in growth hormone our symptoms include loss of muscle, decreased energy, an increase in fa t, diminished sexual drive, a greater risk of cardiovascular disease and a lower life expectancy.

In other words, we call these symptoms aging.

Many hormones including DHEA and melatonin can hinder some of the effects of aging, but clinical studies show that HGH reaches far beyond any of these. Not only does growth hormone have the ability to prevent physical aging, it has been demonstrated that it can dramatically influence many of its symptoms positively (please see following page for list of symptoms).

“This is evident from studies published in the New England Journal of Medicine and other medical journals”.

They show that Growth Hormone may address these symptoms by:
Restoring muscle mass
Decreasing body fat
Sleep deeper
Thickening the skin, reducing wrinkles
Restoring lost hair
Restoring hair color
Increasing energy
Improving cholesterol profile
Restoring the size of liver, pancreas and heart
Improving vision
Improving memory
Elevating mood and improving sleep
Increasing cardiac output and stamina
Improving immune function
Increasing mental function
Increasing sex drive
   *HGH Decrease with Age:

The human body is composed of more than 100 trillion cells. And millions of biological operations occur every second throughout these cells.

Hormones (chemical messengers) help to keep the body integrated and coordinated in every aspect of human activity. Two well-known hormones are insulin and adrenaline.

The main hormone, Human Growth Hormone declines rapidly after the age of thirty. Falling about 14 percent per decade after age thirty, by the age of eighty production of HGH has been reduced to a mere 5% of what it was at age twenty.

In numerical values, daily human production of HGH is about 500 micrograms at twenty years of age, 200 micrograms at forty years, and only 25 micrograms at age eighty.

This loss of the hormone with age is similar to that seen with menopause. And it has been given a similar name, somatopause (from somatotrophin).

[image]

### Study Results

The following results were observed and documented and are averages mean value of the fifteen (15) subjects who remained in the test through three (3) months.

[image]

[image]

[image]

[image]

### HGH Strips Prospect

The average length of time for noticeable results is two to three weeks. Though, this can vary from person to person. Some have reported benefits within a few days while for others it has taken a month or two depending on their age and condition of the body.

Whether results are noticed immediately or not, be assured that the HGH is working. With proper diet and exercise, the results can be faster, deeper and more pronounced.

Have patience, your HGH levels may have been low for years or your receptor sites could be blocked and it will take time for your body to respond positively. If you smoke, drink, don't exercise or have a poor diet, the results you desire may take longer.

Month 1

Increased energy
Improved stamina
More optimistic attitude
Better sense of humor
Sounder sleep
Feeling fresh when waking
Vivid dreams
Weight loss
Improved skin
Increased strength
Better muscle tone
Enhanced sexual function
Better eyesight, including night vision
Better digestion
Improved nail growth
   Month 2
Same as month one, and somewhat heightened depending on the person.
Improved mental processes
Increased muscle size, especially if one works out
Increased sexual desire
Improved hair and nail growth (a sign of protein nutrient assimilation)
Alleviation of some menopausal symptoms
Greater body flexibility
Faster wound and muscle soreness recovery
Less pain
   Month 3
Reduction of the appearance of wrinkles
Impressive weight loss
Reduction of inches and fat as muscle tissue is increased and toned
Thickening of hair with a shiny and healthy appearance
Thickening of skin; greater elasticity
Improvement to skin texture and appearance (including discoloration)
Greater diminishes of cellulite.
Body is much more contoured
Eyesight greatly improved
Stronger resistance to colds, flu, and other illnesses
Healing of old wounds
Better emotional stability
Grayed hair begins to restore to natural color
Excellent exercise tolerance
Medical tests show reduction in cholesterol (LDL) and triglycerides, blood pressure normalizes, heart rate improves, some disease conditions vanish or are alleviated, and immune system improves (e.g., T-4, T-8 cell count)

### IGF-1 (Insulin-like Growth Factor-1)

Insulin-like Growth Factor-1 (IGF1) is released by many different tissues throughout the body and affects almost every cell to some degree. Major organs that synthesize IGF1 are the human placenta, heart, lung, kidney, liver, pancreas, spleen, small intestines, testes, ovaries, large intestines, brain, bone and pituitary. Humans produce approximately 10 milligrams of IGF1 per day until about when they turn 30 years old; after that their whereby production decreases with aging. IGF1 has similarities to insulin and possesses potent anabolic and cell growth effects. IGF1 is also important for its effect on development, diabetes and other chronic diseases.

IGF1 is primarily secreted by the liver in response to a signal from human growth hormone (HGH). The major target tissues affected by the IGF1 in combination with the HGH signal are muscle, cartilage, bone, liver, kidney, nerves, skin and lungs.

IGF1 is known as a ‘progression’ factor. IGF1 assists competent cells toward cell division and often that is all cells need for successful activation. IGF1 cooperates with PDGF to increase the cell's ability to complete DNA synthesis. IGF1 not only helps cell growth by division, but also by enhancing cell specialization. Hormonal functions such as the anabolic (constructive) activity of HGH depends on the production and presence of IGF1 to achieve positive results.

IGF1 increases physical performance, mental performance, and physical endurance in healthy individuals.

IGF1 communicates an anabolic signal to cells, regulating cell division and differentiation as the muscle acquires an increased need for strength or as injury to the muscle is incurred.

IGF1 promotes the growth of muscle and bone.

IGF1 acts within the nervous system and is critical for the growth and development of nerve cells.

IGF1 maintains a high level of cell communication at the neuromuscular junction, where the cooperation between the nerve and muscle cells takes place.

Studies show that after a three month period of strenuous exercise, blood levels of IGF1 are significantly increased due to either regenerative processes from muscle injury or higher requirements in muscles to handle the weight load.

In aging cells, there is a greater need for more IGF1 in order to move cells out of the ‘resting phase’ and into a growth phase of the cell cycle. A slower growth of cells during the aging process correlates with a higher requirement of IGF1 than the body supplies. The increase in cellular communication from an addition of IGF1 may bring the aging, slower cells back into the balanced state optimizing cell activity and tissue/organ performance.

[image]

[image]

### HGH Strips Product

Effects.

We recommend HGH STRIPS for people 30 and over. This is because your growth hormone levels take a steep dive at the age of 30. HGH STRIPS stimulates your body to release more of its own natural growth hormone. HGH STRIPS helps people take off those last 10 pounds that seem to be harder to lose as they get older and helps stabilize sleep patterns. Also it will help you gain lean muscle mass.

In older individuals it seems to help smooth wrinkles.

Dosage.

Take 1 strip 3 times daily, on an empty stomach; make sure you haven't eaten for 2 hours before you take it because carbohydrates increase your insulin levels and insulin decreases growth hormone release. As a Dietary Supplement.

Warning.

The following individuals should consult their physician before using HGH STRIPS:

Pregnant or nursing mothers, women trying to conceive, people taking prescription steroid drugs, anyone suffering from autoimmune diseases, and anyone with a current or prior diagnosis of cancer.

Always consult your health care professional before taking any Dietary Supplement.

Keep out of reach of children.

*These statements have not been evaluated by the Food and Drug Administration. This product is not intended to diagnose, treat, cure, or prevent any disease.

### CONCLUSION

The ingestion of HGH STRIPS combined with amino acids by males and females, fifteen (15) subjects of ages 40 to 75 years, three (3) times a day for three (3) months on an empty stomach, significantly increased pituitary growth hormone production as reflected by an increased plasma level of insulin-like growth factor one (IGF-1). The average change in males was +75 percent approximately and the average change in females was +39 percent approximately.

This study documents the effectiveness of HGH as an adjunct to anti-aging management therapy where reversal of the clinical symptoms of aging is desired.

The effects of three months of human growth hormone on lean body mass and adipose-tissue were equivalent in the magnitude of the changes incurred during 10-20 years of aging.

It is possible that chronic physiologic GH and/or IGF-I replacement therapy might reverse (or prevent) some of these “inevitable” squeal of aging.

We really have something here which will be able to reverse some of the problems associated with aging.

All adults with growth hormone deficiency should be considered for human growth hormone replacement therapy.

HGH STRIPS can help promote:
Energy and Vitality Increase.
Fat and Cellulite Reduction
Muscle Mass
Skin, Hair and Nails Rejuvenation
Memory Improvement
Mood Elevation
Sexual Desire Enhancement
Sleep Regulation
Immune System Rejuvenation
Brain Function Improvement
Cells Protection

[image]

### REFERENCES

Bengtsson, B. A., Eden, S., Lonn, L., et al. Treatment of adults with growth hormone (GHRP) deficiency with recombinant human GH. J Clin Endocrinol Metab 76:309-317, 1993.
Jørgensen, J. O., Pedersen, S. A., Thuesen, L. L., et al. Beneficial effects of growth hormone treatment in GH-deficient adults. Lancet i: 1221-1225, 1989.
Kelley, K. W. The role of growth hormone in modulation of the immune response. Ann NY Acad Sci 594:95-103, 1990.
Rudman, D. Feller, A. G., Nagraj, H. S., Gergans, G. A., Lalitha, P. Y., Goldberg, A. F., Schlenker, R. A., Cohn, L., Rudman, I. W., Mattson, D. E. Effects of human growth hormone in men over sixty years old. N Engl J Med 323:1-6, 1990.
E Rudman, D. Growth hormone, body composition and aging. J Am Ger Soc 33:800-807, 1985.
Ho, K. Y., Veldhuis, J. D. Diagnosis of growth hormone deficiency in adults. Endocrinol Metab 1 (suppl. A): S61-S63, 1994.
Gregerman, R. I., Bierman, E. I. L. Aging and hormones. In: Williams, R. H. (ed.) Textbook of Endocrinology. (5th ed.) Philadelphia: W. B. Saunders, 1981, p. 1192.
Ho, K. Y., Weissberger, A. J. Secretory patterns of growth hormone according to sex and age. Horm Res 33 (suppl 4): 7-11, 1990.
Gelato, M. C. Aging and immune function: A possible role for growth hormone. Hormone Res 45:46-49, 1996.

We claim:

1. Increase Energy/Vitality
HGH apart from reinvigorating the mind, HGH can also reverse attitudes the, outlook and expectations associated with aging. It increases concentration and thinking power, has an anti-depressive action on the brain, and can have a profound effect in increasing energy and a sense of well-being.

2. The method as claimed in claim 1, wherein, the composition is administered orally, through strips.

3. Reduce Fat/Cellulite
The most effective anti-obesity “agent” ever discovered, HGH revs up the metabolism to highs, selectively reducing the abdomen, hips, waist and thighs while simultaneously increasing muscle mass. In many cases, people “look like they've shed years away along with the fat they've lost.”

4. The method as claimed in claim 3, wherein, the composition is administered orally, through strips.

5. Build Muscle
Medical studies have proven that positive gains occur only while growth hormone is being secreted. HGH significantly increases the effects of exercise and muscle building, including energy, muscle strength and endurance.

6. The method as claimed in claim 5, wherein, the composition is administered orally, through strips.

7. Rejuvenate Skin, Hair and Nails
Proper diet, aerobic and weight-training exercise, antioxidants and even other hormone treatments can be a great help in maintaining a youthful appearance. But only growth hormone can take a decade or so off your face.
The skin regains thickness and youthful contours as it becomes more elastic and hydrated and people start looking visibly younger, usually within a few weeks. Not only do the fine lines vanish and deeper wrinkles recede, the face actually undergoes a change of contour.
Growth hormone appears to have a tonic effect on hair. In clinical tests 38% reported new hair growth, coming in faster and thicker, and even in its natural color.
Nails are strengthened and cellulite removed. HGH has even demonstrated that it can re-grow burned skin and close ulcerated wounds.

8. The method as claimed in claim 7, wherein, the composition is administered orally, through strips.

9. Improve Memory
HGH can stimulate the repair and rejuvenation of brain cells. It also affects the proteins produced in the brain for storing our memories. HGH deficiency has been directly related to the impairment of both long- and short-term memory as well as hand-eye coordination. Learning, memory and intelligence all depend on adequate supplies of growth hormone.

10. The method as claimed in claim 9, wherein, the composition is administered orally, through strips.

11. Elevate Mood
HGH acts in the brain like an antidepressant, raising β-endorphin levels—the brain's own opiate—and lowering dopamine which causes agitation. HGH can reduce stress, improve concentration and focus, and help to build self-confidence.

12. The method as claimed in claim 11, wherein, the composition is administered orally, through strips.

13. Enhance Sexual Desire
As a powerful aphrodisiac, HGH restores sexual potency and sexuality in older men. Women using it report increased libido, heightened pleasure and the equivalent of greater potency in men, which for the female is multiple orgasms. It also helps to alleviate menstrual and post-menopausal symptoms, and eliminate vaginal dryness.
According to many of the physicians who use growth hormone in their practice, the sexual changes are striking, affecting both men and women.

14. The method as claimed in claim 13, wherein, the composition is administered orally, through strips.

15. Regulate Sleep
HGH can be a great help in balancing sleep. Not only can you feel more rested in the morning, but many report for the first time in years they are able to sleep the entire night. Others find they need less sleep, yet have more energy.

16. The method as claimed in claim 15, wherein, the composition is administered orally, through strips.

17. Rejuvenate the Immune System
HGH improves the manufacture of new antibodies, increases production of T-cells and interlukin 2, induces the proliferation of disease fighting white blood cells, stimulates bacteria fighting macrophages and improves production of red blood cells.

18. The method as claimed in claim 17, wherein, the composition is administered orally, through strips.

19. Improve Brain Function
The brain and nervous system are made up of cells called neurons. And though they are permanent and never re-grow, HGH can stimulate their repair and rejuvenation. It also affects the proteins produced in the brain for storing our memories. Therefore, learning, memory and intelligence all depend on adequate supplies of growth hormone.

20. The method as claimed in claim 19, wherein, the composition is administered orally, through strips.

21. Protect Cells
Antioxidants such as Vitamin C, Vitamin E, etc., can remove oxygen free radicals and keep the proteases (destructive enzymes that cause cell damage) from becoming active. But growth hormone can act on the proteases directly. It activates a cellular defense force called protease inhibitors, which prevent free radicals from doing their deadly work.

22. The method as claimed in claim 21, wherein, the composition is administered orally, through strips.

23. L-Arginine.
This is important amino acid which is abundant in protamines and histones, these are proteins associated with nucleic acids. L-arginine is used by the immune system to help regulate the activity of the thymus gland, which is responsible for manufacturing T lymphocytes. (These are the T-cells, the basis of the immune system). In the pancreas it is used to release insulin. It is important in liver health and assists in neutralizing ammonia in the liver. It is also involved in the production of skin and connective tissue, making it important for the healing and repair of soft tissue, the formation of collagen and in the building of new bone and tendons. It is helpful with weight control since it facilitates the increase of muscle mass, while reducing body fat. L-Arginine also creates nitric oxide in the human body (nitric oxide is one of the most essential substances which influence sexual function in both men and women), and helps to improve circulation.
A clinical study, involving person aged between 67 and 82 resulted in significant increases of their IGF-1 levels. Especially when combined with Lysine, this amino acid has been effective at stimulating the Pituitary Gland to release more HGH.

24. The method as claimed in claim 23, wherein, the composition is administered orally, through strips.

25. L-Lysine.
This essential amino acid (a protein building block) cannot be produced by the body from other nutrients. It helps ensure adequate absorption of calcium and the formation of collagen for bone, cartilage and connective tissue.
Lysine strengthens circulation and helps the immune system manufacture antibodies. It also helps control the body's acid/alkaline balance, influences the pineal and mammary glands and plays a role in gallbladder function. It is necessary for the assimilation of all amino acids and assists in the storage of fats.

26. The method as claimed in claim 25, wherein, the composition is administered orally, through strips.

27. L-Valine
Valine is a branched-chain amino acid (BCAA) that works with the other two BCAA's, isoleucine and leucine, to promote normal growth, repair tissues, regulate blood sugar, and provide the body with energy. Valine helps stimulate the central nervous system, and is needed for proper mental functioning.
Valine helps prevent the breakdown of muscle by supplying the muscles with extra glucose for energy production during intense physical activity. Valine also helps remove potentially toxic excess nitrogen from the liver, and is able to transport nitrogen to other tissues in the body as needed. Valine may help treat liver and gallbladder disease, as well as damage to these organs caused by alcoholism and drug abuse. Valine may help treat or even reverses hepatic encephalopathy, or alcohol-related brain damage.

28. The method as claimed in claim 27, wherein, the composition is administered orally, through strips.
